(19) United States
(12) Patent Application Publication
(10) Pub. No.: US 2006/0241713 A1
(43) Pub. Date: Oct. 26, 2006

(54) Programmer having retractable antenna

(75) Inventor: Scott M. Freeberg, White Bear Lake, MN (US)

Correspondence Address: GUIDANT PATENT DOCKETING;FAEGRE & BENSON, LLP, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/110,541

(22) Filed: Apr. 20, 2005

(51) Int. Cl. A61N 1/36

(52) U.S. Cl. 607/30; 607/32; 607/60

(57) ABSTRACT

The present invention is an apparatus and method for the automatic extension and retraction of an antenna for a pacemaker programmer. One embodiment of the apparatus includes an antenna in a shaft, a sensor, and a motor. The sensor detects the opening and closing of the display of the programmer and directs the motor to extend or retract the antenna. The present invention allows extension and retraction of the antenna, which allows full utilization of the antenna during use and protection of the antenna when not in use.

### TECHNICAL FIELD

The present invention relates to programmers for pacemakers. More particularly, the present invention is an apparatus and method for pacemaker programmers that automatically extends and retracts the programmer antenna.

### BACKGROUND

Implantable medical devices (“IMD”) include such devices as pacemakers, tachyarrhythmia control devices, drug delivery devices, and nerve stimulators. After implantation, IMDs may need to be monitored and/or programmed by a doctor, nurse, or individual. The monitoring and programming of IMDs can be done periodically by using an external computerized device called a programmer. Programmers are also known as recorders or monitors. The programmer works non-invasively to monitor and report information from the IMD. The programmer can also establish a bi-directional communication link with the IMD that enables the programmer to receive information from the IMD and also to give instructions to the IMD.

Information may be sent from the IMD to the programmer using radio frequency (RF) transmission or other known forms of transmitting a signal. A programmer can, for example, communicate with the IMD using an antenna that extends from the programmer.

The ease of use and effectiveness of the programmer can be limited by the ability of the programmer to receive and transmit the signal. The signal produced by the IMD is not generated with significant power, so the range at which the signal is detectable is fairly short. To help maximize this range, full extension of the antenna is desirable. There is therefore a need automatically extend the programmer antenna to an optimized position during programmer use. There is also a need to automatically retract the antenna when the programmer is not in use to protect it from breakage.

### SUMMARY

The present invention, according to one embodiment, is a programmer for communicating with an implantable medical device. The programmer includes a programmer interface and a display pivotally-coupled to the programmer interface, the display having a closed position wherein the display is generally parallel to the interface and an open position wherein the display extends at a display angle from the interface. The programmer further includes an antenna having a retracted position wherein the antenna is disposed substantially within an antenna shaft and an extended position wherein the antenna is substantially extended from the shaft and an electromechanical motor operatively coupled to the antenna, the motor adapted to drive the antenna between the retracted position and the extended position. The programmer also includes a sensor for detecting the display angle and generating a control signal and a controller located in the interface for receiving the control signal and delivering a drive signal to the electromechanical motor.

Another embodiment of the invention is a programmer for communicating with an implantable medical device, which includes a programmer interface, a display pivotally-coupled to the programmer interface, the display having a closed position and an open position, the display positionable at a display angle selected between the closed position and the open position, a radio-frequency antenna having a retracted position wherein the antenna is disposed substantially within an antenna shaft and an extended position wherein the antenna is substantially extended from the shaft, and an actuation means for causing movement of the antenna between the retracted position and the extended position.

Yet another embodiment of the present invention is a programmer for communicating with an implantable medical device including a programmer interface, a display pivotally-coupled to the programmer interface, the display having a closed position wherein the display is generally parallel to the interface and an open position wherein the display extends at a display angle from the interface, a radio-frequency antenna having a retracted position wherein the antenna is disposed substantially within an antenna shaft and an extended position wherein the antenna is substantially extended from the shaft, a first gear driven by a change in the display angle, and an antenna extender driven by the first gear such that the antenna extender is calibrated to dispose the antenna in the retracted position when the display is closed and in the extended position when the display is open.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 illustrates an implantable medical device programmer with the antenna fully extended.

FIG. 2 is a block diagram illustrating the functional relationship among various components of the programmer, according to one embodiment of the present invention.

FIGS. 3A-3E show schematic views of a programmer in various stages of deployment.

### DETAILED DESCRIPTION

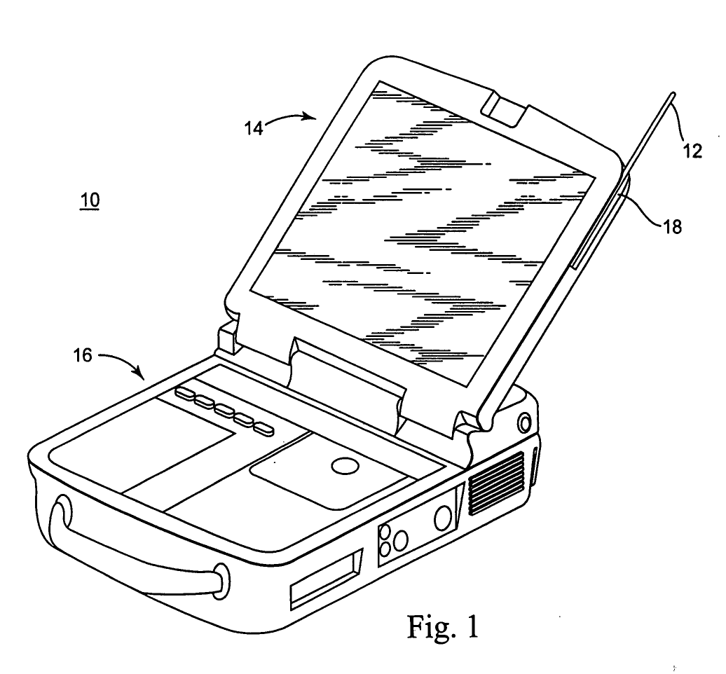

FIG. 1 is a schematic view of an IMD programmer 10 according to an embodiment of the present invention. The programmer 10 may include hand held monitoring devices for various IMDs, including, for example, cardiac pacemakers, neurostimulators, muscle stimulators, brain stimulators, monitoring devices, and other similar devices. As shown in FIG. 1, the programmer 10 includes an antenna 12, a display 14, a programmer interface 16, and an antenna shaft 18. As shown, the antenna 12 is extending at least partially from the antenna shaft 18 located in the display 14. The antenna shaft 18 is sized to at least partially accept the antenna 12, such that antenna 12 may retract partially or fully into the display 14 for protection when not in use. In one embodiment, the antenna 12 is located within a shaft 18 located in the programmer interface 16.

The display 14 may be pivotally coupled to the interface 16 such that it can be rotated from a generally parallel (closed) position to some open position. The antenna 12 may be at any position between a first retracted position and a second extended position. The other components of the programmer 10 can vary depending on the particular design and manufacturer.

The antenna 12 may be a generally rigid rod wherein the whole antenna 12 is linearly extended as a unit. The antenna 12 may also be a rigid rod that swings out from the programmer 10 in an arc rather than extending in a straight line. In other embodiments, the antenna 12 can be collapsible or telescoping. The use of the term “rigid” means a non-collapsible antenna 12. An antenna 12 may be a solid bar of metal or may be a helical wound antenna 12 of a fixed length. In alternative embodiments, the antenna 12 may be extensible, bendable, collapsible, partially rigid, or any other format known to those in the art. In one embodiment, the antenna 12 is adapted to allow RF communication with an IMD.

The display 14 may be any digital display known in the art useful for showing information. The interface 16 may include a keyboard, switch, and/or other types of interface systems, such as a point and click interface. The interface 16, in one embodiment, also includes a drive for accepting magnetic or optical media.

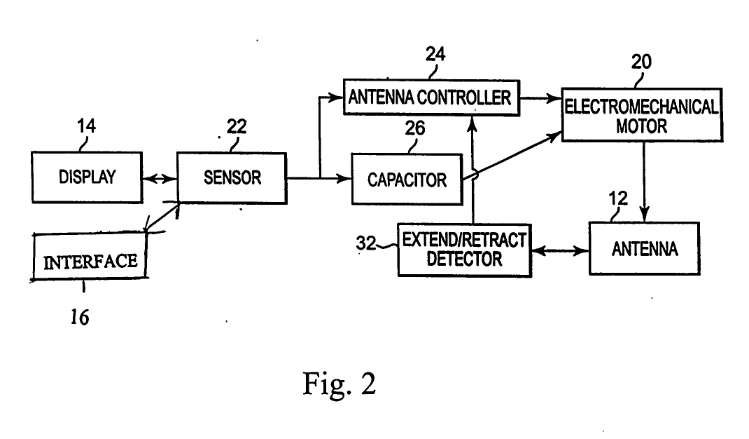

FIG. 2 is a block diagram showing the functional relationship among various components of the programmer 10, according to one embodiment of the invention. As shown, programmer 10 may further include (in addition to those components shown in FIG. 1) an electromechanical motor 20, a sensor 22, and an antenna controller 24. The electromechanical motor 20 may be a drive apparatus for the deployment of the antenna 12. The electromechanical motor 20 may be positioned next to the shaft 18 such that it is operatively connected to the antenna 12. Depending on what type of antenna 12 is present (i.e., extensible, collapsible, swing, etc.), the electromechanical motor 20 may have different gears and other movement or actuation means, such as a screw drive.

The sensor 22 may be positioned such that it detects the movement of the display 14 in relation to the interface 16. The sensor 22 may be incorporated into the display 14 or interface 16. The antenna controller 24 is in electrical communication with the sensor 22 and the electromechanical motor 20. The antenna controller 24 may be integrated into the sensor 22 or may be a separate component operatively and electrically connected to the sensor 22. The antenna controller may be any type of controller known in the art, including, for example, a processing element such as a microprocessor. In one embodiment, the controller is implemented using logic circuitry (e.g., discrete components or programmable logic arrays). The sensor 22 may include a variety of sensors.

During operation of the present invention, the antenna controller 24 controls the position of the antenna 12, based on a control signal from the sensor 22. In one embodiment of the present invention, the sensor 22 is a basic on/off switch. In this embodiment, the sensor 22 detects the display 14 at or above a pre-selected angle relative to the interface 16 and communicates or provides a control signal to the antenna controller 24. The controller 24 then generates a drive signal to activate the motor 20, which operates to extend or retract the antenna 12. Likewise, when the display 14 is closed past a certain angle relative to the programmer 16, the sensor 22 may provide a closed signal to the antenna controller 24. The antenna controller 24 may then cause the electromechanical motor 20 to retract the antenna 12. In various embodiments, the control signal provided by the sensor 22 is a resistance value as measured by the controller 24. For example, where the sensor 22 is a bipolar switch, the control signal is an indication of whether electrical contacts are open or closed.

Alternatively, the sensor 22 may be configured to continuously generate a signal indicating the angle of the display 14 with respect to the programmer 16. The antenna controller 24 monitors this signal and generates a drive signal for the motor 20, when the signal indicates an angle above a predetermined activation angle. For example, in one embodiment the activation angle is set at 90 degrees relative to the display. In another embodiment the activation angle is set at 110 degrees relative to the display.

In one embodiment, the sensor 22 is a bipolar switch. In this embodiment, the sensor provides a control signal that only indicates to the controller 24 whether the display angle is less than or greater than a predetermined value. As mentioned above, this predetermined value may be, for example, 90 degrees or 110 degrees. In this embodiment, the controller 24 generates a drive signal that causes the motor 20 to fully extend or fully retract the antenna 12.

Another sensor 22 used to detect the angle of the display 14 may be a mercury switch mounted in the display 14. A mercury switch is a position-oriented or detection-type of switch. When the liquid mercury flows to one end of the switch capsule, the electrically conductive mercury causes a set of contacts to be closed. When the mercury flows away from the contacts, the contacts open. When the mercury switch is placed in the display 14 at a particular angle, the state of the switch will change when the minimum desired operating angle is reached or exceeded. When the display 14 is opened and passes the minimum angle the mercury switch changes state and indicates to the antenna controller 24 to extend or retract the antenna 12.

Another sensor 22 that may be used to detect the angle of the display 14 is a mechanical switch mounted to the programmer 10 or the display 14 and set to be activated by an actuator or offset cam mounted in an operable position. As the display 14 is rotated open, or when a minimum angle is reached or exceeded, the actuator or offset cam presses against and thus activates the mechanical switch. This activation of the mechanical switch informs the antenna controller 24 that the display 14 is open and to extend the antenna 12. The actuator may continue to press against the switch for as long as the display 14 is above the minimum angle. When the display 14 is lowered past the minimum angle, the actuator mounted to the display 14 will no longer press against the mechanical switch. The switch detects the change and informs the antenna controller 24 to retract the antenna 12. Another sensor 22 used to detect the angle of the display may be a magnetic switch and a magnet.

Yet another sensor 22 may be an indicator from the programmer software application itself. In one embodiment, the display 14 may be opened to start a software application. When the application is opened it would send a signal to the sensor 22 which would indicate to the antenna controller 24 to extend the antenna 12. When the application is closed the antenna controller 24 would retract the antenna 12 based on the signal from the sensor 22.

In one embodiment, the programmer 10 may also include a capacitor 26. The capacitor 26 may provide power to the antenna controller 24 and/or electromechanical motor 20 to retract the antenna 12 in case the power to the programmer 20 fails or is interrupted. The capacitor 26 may charge when the main power source to the programmer 10 is properly attached to the power source. The capacitor 26 can be set to provide power to the controller 24 and the electromechanical motor 20 to lower the antenna 12 into the shaft 18 whenever the programmer 10 is switched off or the power is interrupted, even if the display 14 is still in the open position. Moreover, the antenna 12 and electromechanical motor 20 can also be set such that the antenna does not extend from shaft 18 until the programmer 10 power is activated, even if the display 14 has already been opened.

In one alternative embodiment, the sensor 22, antenna controller 24, and electromechanical motor 20 may be set to move the antenna 12 between certain set positions relative to the position of display 14. As the display 14 is opened past certain predetermined angles, the antenna 12 may be extended to certain set positions.

In alternative embodiments, the sensor 22 may be a linear device such as a resistive potentiometer. In this embodiment, the distance the antenna 12 is extended may be directly and proportionately linked to the angle of the display 14 with respect to the programmer 16. The more that the display 14 is opened, the further the antenna 12 extends. The more that the display 14 is closed, the further the antenna 12 retracts.

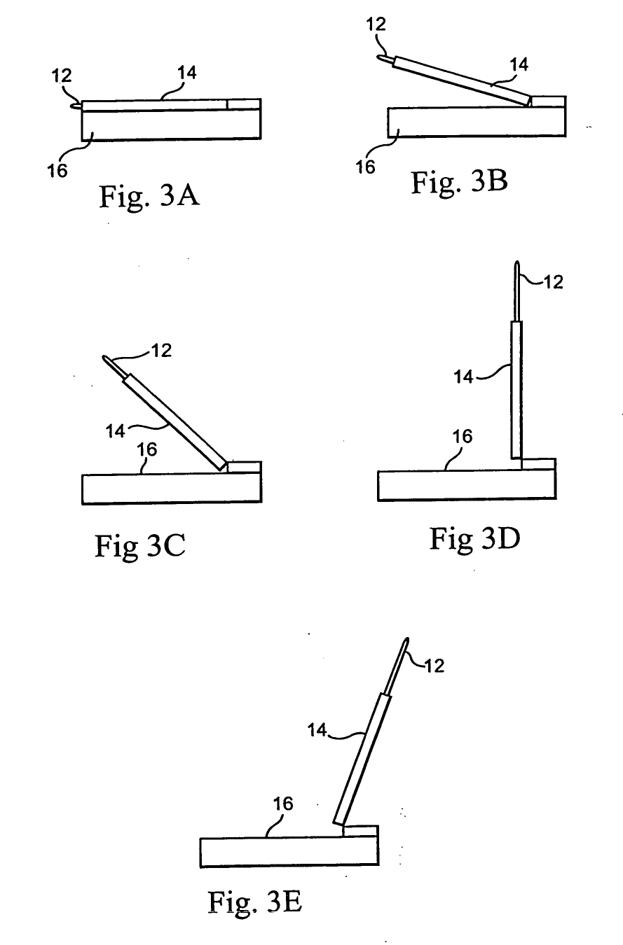

FIGS. 3A-3E show schematic views of a programmer in various stages of deployment. As illustrated, as the relative position of the display 14 changes as compare to the interface 16, the antenna 12 extends or retracts. To achieve such a system, the resistive potentiometer sensor 22 may continuously send a signal to the antenna controller 24. The controller 24, in turn, continuously generates a drive signal to the motor 20 to update the position of the antenna 12.

In another alternative embodiment, the programmer 10 may include a detector 32, which detects the extension or retraction of antenna 12 and signals this information to the antenna controller 24. The controller 24 then uses this information in conjunction with the signal from the sensor 22 to generate an appropriate drive signal to the motor 20. In still a further embodiment, a switch may be included to override the system and automatically extend or retract the antenna 12. The override switch may allow the display 14 to be opened, while the antenna 12 remains in the closed position.

In another alternative embodiment, an apparatus that mechanically extends the antenna 12, as opposed to electromechanically, may replace the electromechanical motor 20. The mechanical extension device would employ a series of gears that are linked to and driven by the opening and closing of the display 14. The act of opening the display 14 would drive the antenna 12 into an extended position and the act of closing the display 14 would retract the antenna 12 into a closed position. The gears can be calibrated such that the antenna 12 is in the full extension position when the display 14 is fully opened position and in the full retraction position when the display 14 is in the fully closed position.

Various modifications and additions can be made to the exemplary embodiments discussed without departing from the scope of the present invention. Accordingly, the scope of the present invention is intended to embrace all such alternatives, modifications, and variations as fall within the scope of the claims, together with all equivalents thereof.

I claim:

1. A programmer for communicating with an implantable medical device, the programmer comprising:
a programmer interface;
a display pivotally-coupled to the programmer interface, the display having a closed position wherein the display is generally parallel to the interface and an open position wherein the display extends at a display angle from the interface;
an antenna having a retracted position wherein the antenna is disposed substantially within an antenna shaft and an extended position wherein the antenna is substantially extended from the shaft;
an electromechanical motor operatively coupled to the antenna, the motor adapted to drive the antenna between the retracted position and the extended position;
a sensor for detecting the display angle and generating a control signal; and
a controller located in the interface for receiving the control signal and delivering a drive signal to the electromechanical motor.

2. The programmer of claim 1 wherein the shaft is located in the display.

3. The programmer of claim 1 wherein the drive signal is delivered when the display angle is at least about 90 degrees with respect to the interface.

4. The programmer of claim 1 wherein the system further comprises an override switch that delivers an override signal to the controller, and further wherein the override signal causes the controller to deliver a drive signal to cause the motor to move the antenna to either the retracted position or the extended position.

5. The programmer of claim 1 further comprising a capacitor electrically coupled to the electromechanical motor.

6. The programmer of claim 1 wherein the sensor is a bipolar switch that generates a bipolar control signal.

7. The programmer of claim 1 wherein the sensor continuously adjusts the control signal based on the display angle.

8. The programmer of claim 7 wherein the sensor is a resistive potentiometer that continually detects the display angle, and further wherein the control signal generated by the resistive potentiometer is proportional to the display angle signal, such that an extension amount of the antenna is proportional to the display angle.

9. The programmer of claim 7 wherein the antenna is fully extended when the display angle is about 110 degrees.

10. A programmer for communicating with an implantable medical device, the programmer comprising:
a programmer interface;
a display pivotally-coupled to the programmer interface, the display having a closed position and an open position, the display positionable at a display angle selected between the closed position and the open position;
a radio-frequency antenna having a retracted position wherein the antenna is disposed substantially within an antenna shaft and an extended position wherein the antenna is substantially extended from the shaft; and
an actuation means for causing movement of the antenna between the retracted position and the extended position.

11. The programmer of claim 10 wherein the actuation means comprises a sensor for detecting a pre-selected angle of the display with respect to the interface and generating a control signal, and a controller located in the interface for receiving the control signal and delivering a drive signal to an electromechanical motor.

12. The programmer of claim 11 wherein the sensor continuously adjusts the control signal based on the display angle.

13. The programmer of claim 11 wherein the sensor is a resistive potentiometer that generates an angle signal that is proportional to the display, and further wherein the control signal is proportional to the display angle signal, such that an extension amount of the antenna is proportional to the display angle.

14. The programmer of claim 10 wherein the actuation means comprises a mechanical linkage coupled to the interface, the display, and the antenna, the mechanical linkage configured to cause extension of the antenna in response to an increase in the display angle.

15. The programmer of claim 11 further comprising an override switch adapted to deliver an override signal to the controller to prevent the controller from generating a drive signal.

16. A programmer for communicating with an implantable medical device, the programmer comprising:
a programmer interface;
a display pivotally-coupled to the programmer interface, the display having a closed position wherein the display is generally parallel to the interface and an open position wherein the display extends at a display angle from the interface;
a radio-frequency antenna having a retracted position wherein the antenna is disposed substantially within an antenna shaft and an extended position wherein the antenna is substantially extended from the shaft;
a first gear driven by a change in the display angle; and
an antenna extender driven by the first gear such that the antenna extender is calibrated to dispose the antenna in the retracted position when the display is closed and in the extended position when the display is open.

17. The programmer of claim 16 wherein the antenna is mounted in a shaft in the display.

18. The programmer of claim 16 wherein the antenna is mounted in a shaft in the interface.

19. The programmer of claim 16 wherein the antenna is fully extended when the display angle is about 90 degrees.

20. The programmer of claim 16 wherein the antenna is fully extended when the display angle is about 120 degrees.
